(19) United States
(12) Patent Application Publication
(10) Pub. No.: US 2014/0257045 A1
(43) Pub. Date: Sep. 11, 2014

(54) HIERARCHICAL EXPLORATION OF LONGITUDINAL MEDICAL EVENTS

(71) Applicant: INTERNATIONAL BUSINESS MACHINES CORPORATION, Armonk, NY (US)

(72) Inventors: Jianying Hu, Bronx, NY (US); Adam N. Perer, Long Island City, NY (US); Fei Wang, Ossining, NY (US)

(73) Assignee: INTERNATIONAL BUSINESS MACHINES CORPORATION, Armonk, NY (US)

(21) Appl. No.: 13/790,021

(22) Filed: Mar. 8, 2013

(51) Int. Cl. A61B 5/00

(52) U.S. Cl. CPC A61B 5/7282; A61B 5/742
USPC 600/300

(57) ABSTRACT

Systems and methods for data analysis include determining medical events co-occurring within a time period from a patient record database. The medical events are grouped into sets of medical events such that a number of sets of medical events is minimized based upon medical event cardinality. Patterns from the sets of medical events are identified, using a processor, to provide relationships between the patterns and patient outcomes.

### BACKGROUND

1. Technical Field

The present invention relates to analysis of electronic medical records, and more particularly to the hierarchical exploration of longitudinal medical events.

2. Description of the Related Art

Temporal analysis of Electronic Medical Records (EMR) is an important problem in medical informatics as the sequences of medical events often have clinical significance. Identifying such sequences can lead to better identification and prediction of disease condition of patients, as well as discovery of treatment action or sequence of actions that lead to better outcomes. Common approaches to temporal analysis of EMR are based on Business Process Management (BPM) techniques to summarize traces of patient populations with care pathway models. However, as there is a high degree of variability on the behavior and treatments of individual patients, the pathway models determined via BPM are usually highly complex and difficult to understand and interpret. As such, implementing results from such approaches is difficult.

### SUMMARY

A method for data analysis includes determining medical events co-occurring within a time period from a patient record database. The medical events are grouped into sets of medical events such that a number of sets of medical events is minimized based upon medical event cardinality. Patterns from the sets of medical events are identified, using a processor, to provide relationships between the patterns and patient outcomes.

A system for data analysis includes a data preprocessor configured to determine medical events co-occurring within a time period from a patient record database and group the medical events into sets of medical events such that a number of sets of medical events is minimized based upon medical event cardinality. A frequent pattern analysis engine is configured to identify patterns from the sets of medical events to provide relationships between the patterns and patient outcomes.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of a system/method for hierarchical information exploration, in accordance with one illustrative embodiment;

FIG. 2 is a block/flow diagram showing a structure of a patient electronic medical records dataset, in accordance with one illustrative embodiment;

FIG. 3 shows a hierarchical branch for the hierarchy cardiac disorders, in accordance with one illustrative embodiment;

FIG. 4 is a hierarchical branch for the pharmacy class beta blockers, in accordance with one illustrative embodiment;

FIG. 5 shows a graphical illustration of breaking down concurrent medical events, in accordance with one illustrative embodiment;

FIG. 6 shows an exemplary visual interface, in accordance with one illustrative embodiment; and

FIG. 7 is a block/flow diagram showing a system/method for hierarchical information exploration, in accordance with one illustrative embodiment.

### DETAILED DESCRIPTION OF PREFERRED EMBODIMENTS

In accordance with the present principles, systems and methods for hierarchical exploration of longitudinal medical events are provided. A patient record database is provided, which may include electronic medical records hierarchically arranged according to medical event. Medical events co-occurring within a time period from a patient record database are identified (e.g., Same Day Concurrent Events (SDCEs)). The SDCEs are grouped into sets of medical events such that the number of sets is minimized. In a preferred embodiment, medical event packages are identified and the medical event package with a highest cardinality is provided as a set. Where there are multiple medical event packages that have the highest cardinality, the medical event package with a highest appearance frequency is provided as the set. This process is repeated for remaining portions of the SDCE.

Patterns are identified from the sets of medical events to provide relationships between patterns and patient outcomes. This may include employing frequent pattern mining techniques. Patterns may be arranged in a pattern dictionary and bag-of-pattern representations may be constructed to further enable outcome analysis.

Relationships between the patterns and patient outcomes may be displayed, where medical events are represented as nodes and nodes of medical events belonging to a same pattern are connected by edges. The edges may be represented by patient outcome (e.g., by color, etc.). Advantageously, the selection of nodes and/or edges are enabled to allow users to explore the list of patients or patterns in more detail, in a hierarchical manner.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

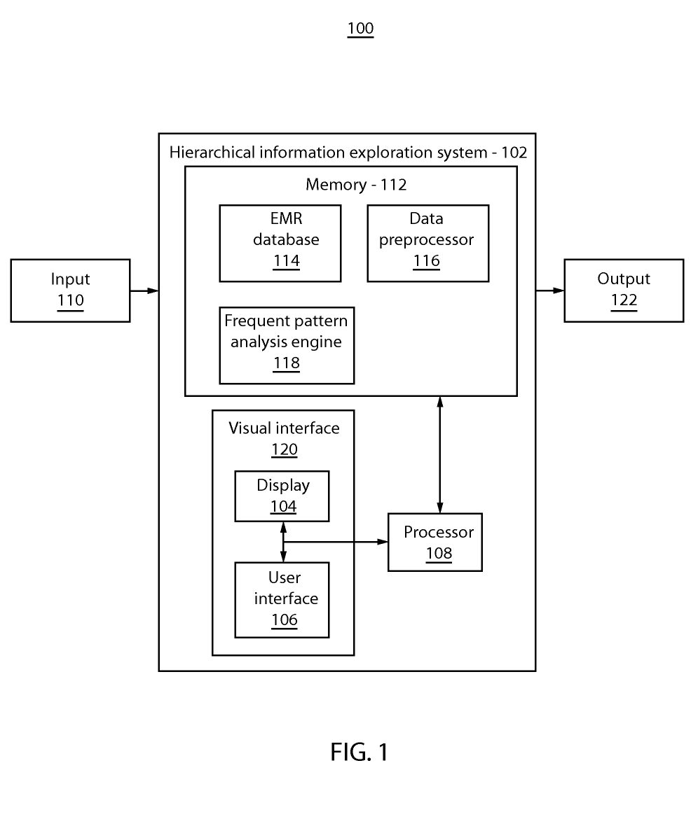

Referring now to the drawings in which like numerals represent the same or similar elements and initially to FIG. 1, a block/flow diagram showing a hierarchical information exploration system 100 is illustratively depicted in accordance with one embodiment. The system 100 may analyze data, such as, e.g., patient longitudinal data, to provide a visual overview of frequent patterns determined from the patient traces. The system 100 thus supports interactive exploration for physicians or clinical researchers to examine the level-of-detail of interest.

The system 100 may include a system or workstation 102. The system 102 preferably includes one or more processors 108 and memory 112 for storing applications, modules and other data. The system 102 may also include one or more displays 104 for viewing. The displays 104 may permit a user to interact with the system 102 and its components and functions. This may be further facilitated by a user interface 106, which may include a mouse, joystick, or any other peripheral or control to permit user interaction with the system 102 and/or its devices. It should be understood that the components and functions of the system 102 may be integrated into one or more systems or workstations.

System 102 may include an input 110, which may include constraints for viewing patient event traces, patient medical records stored in Electronic Medical Record (EMR) database 114, etc. EMRs are a systematic collection of longitudinal patient health information generated by encounters in care delivery settings. EMR data may include, e.g., patient demographics, as well as encounter records such as claims, progress notes, problems, medications, vital signs, immunizations, laboratory data, radiology reports, etc. EMR database 114 stores the patient medical records with multiple event types along with the actual patient outcomes.

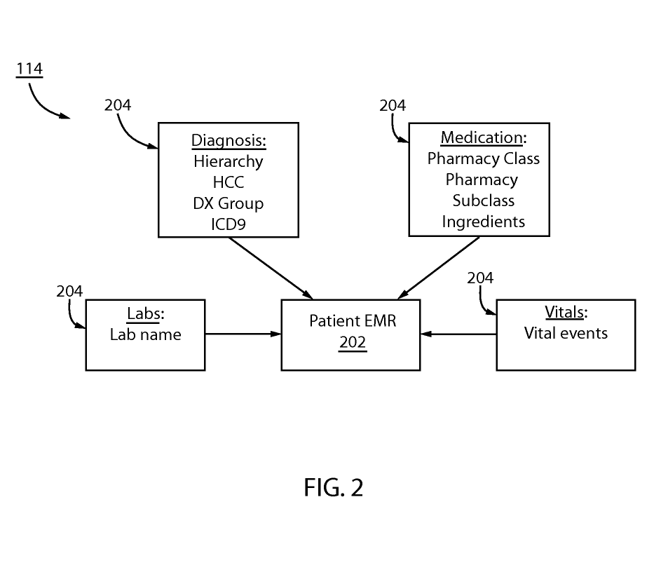

Referring for a moment to FIG. 2, a structure of EMR database 114 is illustratively depicted in accordance with one embodiment. EMR database 114 illustrated in FIG. 2 is used for predicting hospitalization for congestive heart failure (CHF). EMR database 114 may include patient EMR 202 and events 204. Events 204 may include medical events, such as, e.g., lab, vital, medication and diagnosis. Other events are also contemplated. In a preferred embodiment, EMR database 114 is stored in a relational model database server, such as, e.g., IBM's DB2 database, as a Universal Feature Model (UFM), which may include a four column table indicating patient ID, day ID, event ID and an event value. The diagnosis and medication events may include a defined hierarchy, illustrated in the following Tables 1 and 2 in accordance with exemplary embodiments. The events are restricted to be medically relevant diagnoses and medications to CHR or its co-morbidities in this illustrative embodiment.

TABLE 1
Exemplary diagnosis hierarchy information

| Level Name | # Events |
|---|---|
| Hierarchy Name | 3 |
| Hierarchical Condition Categories (HCC) Code | 4 |
| DX Group Name (first three digits of ICD9 code) | 10 |
| International Classification of Diagnosis 9th Edition | 42 |
| (ICD9) Code | |

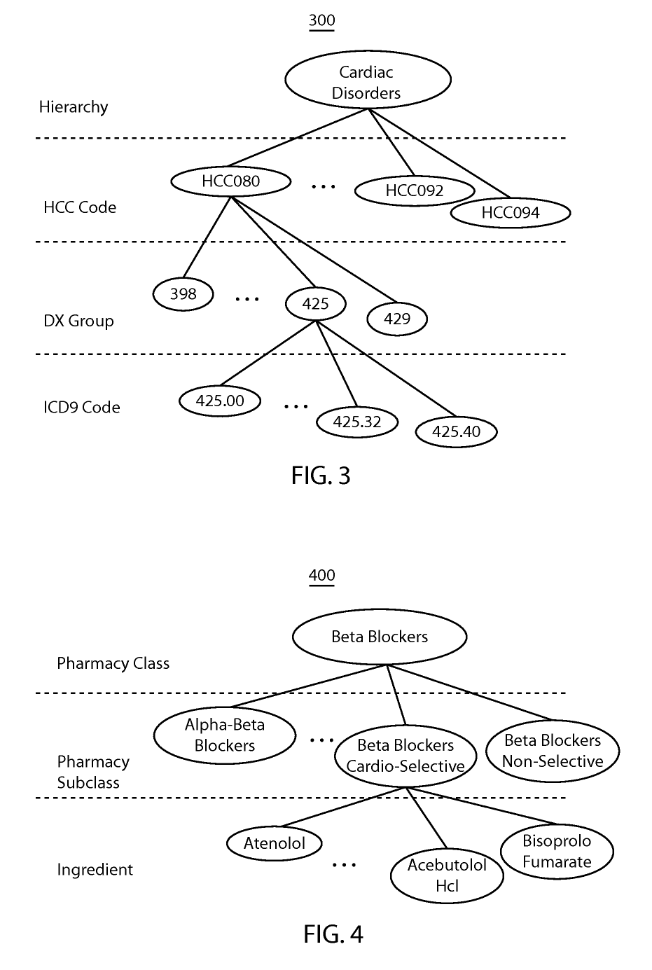

The diagnosis hierarchy may include four levels, as illustrated in Table 1. The first level is the hierarchy name, which includes three distinct values. The second level is a Hierarchical Condition Categories (HCC) code, which includes four different values. The third level includes 10 unique Diagnosis (DX) group names. The fourth level includes 42 different codes of the International Classification of Diagnosis 9th Edition (ICD9). Each level in this diagnosis hierarchy is a many-to-one mapping. That is, each node in a specific level includes one or more nodes in one level lower. FIG. 3 illustratively depicts a branch of the hierarchy 300 for the hierarchy Cardiac Disorders, in accordance with one embodiment.

TABLE 2
Exemplary medication hierarchy information

| Level Name | # Events |
|---|---|
| Pharmacy Class | 6 |
| Pharmacy Subclass | 18 |
| Ingredients | 66 |

The medication hierarchy may include four levels, as illustrated in Table 2. The levels may include pharmacy class, pharmacy subclass and ingredient, from the highest to lowest level. Table 2 summarizes an exemplary number of distinct events on each level. FIG. 4 illustratively depicts a branch of the hierarchy 400 for the pharmacy class beta blockers, in accordance with one embodiment.

Data preprocessor 116 may be configured to construct a set of patient traces from EMR database 114. The finest resolution of the temporal data in EMR database 114 is, e.g., a day, and during a day, multiple medical events typically occur for a patient. Such data characteristics yields a great challenge for existing frequent pattern mining approaches, as they detect patterns with all possible combinations of events and subsets of events occurring at the same time. For example, consider the frequent pattern (A;B→A;C). Then, (A→A), (A→C), (A;B→A), (A;B→C), (A→A;C), and (B→A;C) are all frequent patterns (note: a semicolon connotes events occurring at the same time). If there are even more concurrent events, the number of detected frequent patterns increases dramatically. This phenomenon is referred to as pattern explosion.

To address pattern explosion, patient traces are preprocessed before performing frequent pattern mining (in frequent pattern analysis engine 118). Patient EMRs include many same day concurrent events (SDCEs). Thus, the frequent Clinical Event Packages (CEPs), which are subsets of events that frequently occur among all SDCEs, are first detected (e.g., using Frequent Itemset Mining). It is noted that the present principles are not limited to concurrent events occurring on the same day; other time periods are also contemplated. If each SDCE in every patient trace is treated as a transaction, the problem is similar to frequent itemset mining and each detected clinical event package can be used as a super event.

A greedy approach may be applied based on Two-Way Sorting to break down each SDCE as a combination of regular and super events to significantly reduce the number of events contained in each SDCE. First, CEPs identified in a SDCE are sorted according to their cardinalities. Then, CEPs with a same cardinality are sorted based on frequency of appearance. The CEP with the highest cardinality is selected as a superevent. If there are multiple CEPs with the highest cardinality, the CEP with a highest frequency of appearance is selected as a superevent. The process is repeated for the remaining CEPs of the SDCE.

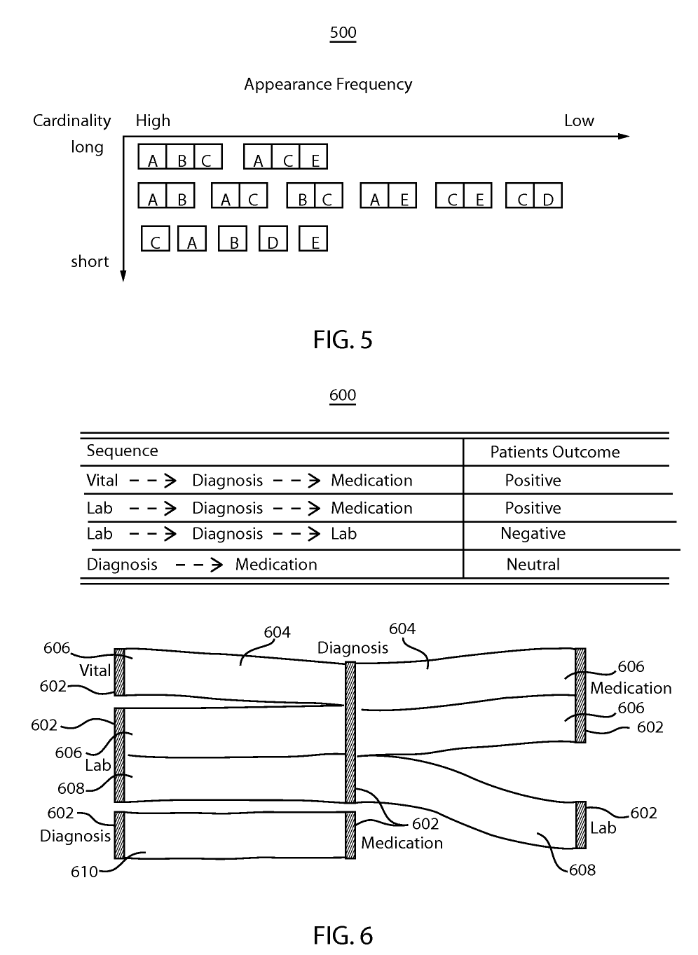

Referring now to FIG. 5, a graphical illustration 500 of breaking down SDCEs is illustratively depicted in accordance with one embodiment. Supposed the SDCE ABCDE is to be broken down based on the detected Clinical Event Packages (CEPs). The packages are sorted according to the two-way sorting strategy, as illustrated in FIG. 8. First, packages are sorted according to their cardinalities. Then, packages with the same cardinality are sorted with respect to their appearance frequency. To breakdown ABCDE, the two-way sorting strategy finds the longest clinical packages that are subsets. In this case, ABC and ACE are the longest packages, which are subsets of ABCDE. Then, because ABC occurs more frequently than ACE, ABC is selected as a super event contained in ABCDE. The remaining events are DE. Then the procedure is repeated to break down DE into the super events D and E. The breakdown of ABCDE is found to be ABC, D, E. Using this technique, there are only 3 super events in ABCDE, as opposed to having 5 events.

Pseudocode 1 summarizes the main procedure of breaking down a specific SDCE. Note that after the sorting procedure in line 1, all of the CEP buckets are ordered from the largest cardinality to the lowest. After the sorting procedure in line 2, all CEPs within each bucket are ordered from the highest frequency to the lowest. The enumeration process of all buckets and CEPs in lines 4 and 6 are according to these orders.

Pseudocode 1: illustrative example of breaking down SDCEs, in accordance with one embodiment.

Input: An SDCE S to be broken down, Detected Clinical Event Packages
(CEP)
1: Sort the detected CEPs into buckets according to their cardinalities
(number of events contained), such that the packages within the same
bucket have the same cardinality.
2: Sort the packages within the same bucket with their appearance
frequencies in the patient traces.
3: O = 0 ;
4: for Every bucket B do
5: if length(B) < length(S) then
6: for Every CEP ε in B do
7: if ε is a subset of s then
8: Add ε to O, Set S = S \ ε
9: if S == 0 ; then
10: Return O
11: else
12: Return to Line 4
13: end if
14: end if
15: end for
16: end if
17: end for

Frequent pattern analysis engine (FPAE) 118 is configured to perform frequent pattern mining on the broken down events from data preprocessor 116. FPAE 118 identifies frequent patterns from patient traces obtained by the data preprocessor 116 and analyzes how the patterns correlate with outcomes. Frequent patterns are patterns (i.e., subsequences) that occur frequently in a dataset. Preferably, the FPAE 118 applies the SPAM (Sequential Pattern Mining) technique for frequent pattern mining, as it adopts a smart depth-first search strategy and is more efficient for mining patterns from long sequences. Other frequent pattern techniques may also be employed.

After applying frequent pattern analysis to detect frequent patterns, patterns are collected into a pattern dictionary, which is a set of frequent event subsequences that are detected from the entire patient population. A Bag-of-Pattern (BoP) representation, which may include a vector, for each patient trace is constructed. Suppose the pattern dictionary size is m, then the BoP vector for each patient is an m-dimensional vector, such that the value on the i-th dimension represents the frequency of the i-th pattern in the corresponding patient trace. When counting pattern frequency, the bitmap representation of patient trace is applied and pattern matching is done bit by bit. Ultimately, the pattern frequency is the number of matches.

This BoP representation can further enable outcome analysis, where patterns are the features and the patient traces are the data. Each patient can be associated with an outcome, which can be discrete (e.g., deceased vs. alive) or continuous (e.g., HbA1c value for diabetes patients). The pattern can be analyzed to determine whether it has an impact on outcomes using feature selection techniques.

The system 102 may provide a visual interface 120, which may be included in output 122. Visual interface 120 may involve display 104 and/or user interface 106 to illustrate relationships between frequent patterns and outcomes and allow user interaction to explore details of interest and generate insights. The relationship between frequent patterns and outcomes can be used to understand disease evolution and optimize treatments. However, the quantity of patterns discovered is often too large for users (e.g., doctors) to make sense of them. Thus, system 102 provides a visual interface 120 to present the data is a user-centric way so that patterns can be utilized in real-world settings. Information visualization is an effective way of communicating complex data, and thus, an important component of the visual interface 120 of the system 102 is flow visualization.

Referring for a moment to FIG. 6, an exemplary visual interface 600 of the system 102 for a set of frequent patterns is illustratively depicted in accordance with one embodiment. Events in the frequent patterns are represented as nodes 602, and nodes 602 that belong to the same pattern are connected by edges 604. For instance, the pattern (Diagnosis→Medication) is visualized as a Diagnosis node connected to a Medication node in FIG. 6. Patterns that share similar subsequences, such as (Lab→Diagnosis→Medication) and (Lab→Diagnosis→Lab), involve two edges from Lab to Diagnosis representing each subsequence. Thus, prominent subsequence patterns also become visually prominent due to the thickness of the combined multiple edges.

Not all patterns are equal, as some correlate to good outcomes for patients whereas others correlate to bad outcomes. Visual interface 120 visually encodes each pattern's association with outcome (i.e., positive, negative or neutral). In a preferred embodiment, the outcome of a pattern may be associated with a color. Edges indicating a positive patient outcome 606 (e.g., those who are not hospitalized within the first year of diagnosis) may be colored blue. Edges indicting a negative patient outcome 608 (e.g., those who are hospitalized within the first year after diagnosis) may be colored red. Edges indicting a neutral patient outcome 610 (i.e., patterns that appear common to both negative and positive patients) may be colored gray. It is noted that other visual encodings may also be applied within the scope of the present principles, such as, e.g., patterns, etc. Users may be about to mouse-over edges to get additional data, including, e.g., a description of the pattern and statistics describing the patients.

Visual interface 120 may be organized hierarchically, in harmony with the EMR database 114. Initially, visual interface 120 is populated with an overview of all frequent patterns at the coarsest level. This overview visualization acts as starting points for users to interact with the visualization and explore patterns of interest. Users may click a sequence of nodes or edges to highlight an interesting pattern. This selection enables a query for all patients who have traces that fit this pattern. Users can explore the list of patients, or explore their patterns in more detail by drilling-down to the next level of hierarchy to get more specific information. For instance, if a user selected the pattern (Diagnosis→Medication), the visualization would show all of the patients that matched the pattern, and their pathways would be visualized in more detail using diagnosis HCC codes and medication Pharmacy Subclasses. The user can make selections and hierarchically drill down until the desired level-of-detail is reached.

The visual design of visual interface 120 may appear similar to a sankey diagram. However, sankey diagrams focus on the flow of resources and ignore the sequential ordering, which is a very important feature of EMR data. The Outflow visualization technique may also appear visually similar. However, Outflow aggregates subsequences and outcomes. In the visual interface 120, each frequent pattern (i.e., subsequence) is represented as an individual edge to provide a true overview of all sequences and their individual outcomes. Furthermore, visual interface 120 supports hierarchical navigation.

To better illustration the operation of hierarchical information exploration system 102, an exemplary real-world case study of congestive heart failure (CHF) will be discussed implementing system 102, in accordance with one embodiment. A data warehouse of longitudinal CMR data of around 7 years and 50,000 patients is used. The different types of medical event information in the database and their associated hierarchies are as discussed with respect to EMR database 114 above. The goal of this case study is to utilize this data to investigate the issue of care planning: what are the key care operations that may lead to hospitalization?

To conduct the empirical study, the EMRs for the CHF case patients is extracted beginning with their operational criteria date (i.e., the date of diagnosis with CHF) to either one year after or their first hospitalization date, whichever comes first. The outcomes associated with the patients is binary (hospitalized or not within one year after CHF diagnosis). Positive patients are referred to as those who are not hospitalized within one year after diagnosis, while negative patients are referred to those who are hospitalized within one year of diagnosis. A cohort of 1313 CHF case patients were used in this study, among which 518 are positive patients and 795 are negative patients.

The hierarchical information exploration system 102 was deployed to explore frequent patterns from patient traces with different hierarchy levels of event details. In this data warehouse, three levels of event hierarchies are used: Level 0 is the coarsest level, where there are four different event types: medication, lab, diagnosis and vital. Level 1 has more detailed information on diagnosis (HCC codes) and medications (Pharmacy Class). For medications, the numbers following the pharmacy class name describe the functional classification of the New York Heart Association, numbering 1 to 4 from least to most severe disease condition. On Level 2, there are also concrete names for lab tests. After those patterns are determined, FPAE 118 of system 102 constructs a BoP matrix for the matched patients and computes the Odds Ratio for each pattern. A high odds ratio means the corresponding pattern appears more in positive patients, while a low odds ratio indicates the pattern appears more in negative patients.

System 102 provides visual interface 120 to depict relationships of the frequent patterns. For Level 0, frequent patterns are shown for the four event types: medication, lab, diagnosis and vital. For example, after a lab test, the next step for many patients is vital (which suggests a primary care physician) or diagnosis (which may be from physicians or specialists). After a vital event, the next step may be evenly distributed to medication, lab and diagnosis based on suggestions made by the primary care physician. The patterns may be colored blue to indicate a better management of the disease.

The user (e.g., physician) may then interact with the visual interface 120 to select a subpath (medication→vital→medication→vital) to see more details about this patient sub-cohort who exhibit this pattern. System 102 then queries the database and retrieves the patterns of those patients of Level 1. Visual interface 120 may show that the detailed medications are Beta Blockers 2 and Diuretics 3, and detailed diagnoses are HCC080 (CHR) and HCC091 (hypertension). The visualization also communicates that the pattern flows with HCC091 and Beta Blockers 2 are positive patients (blue) since hypertension is regarded as the most common risk factor of CHR, and Beta Blockers are particularly useful for the management of heart attacks and hypertension. This suggests that effective management of hypertension is of crucial importance to treat CHF patients.

Seeking even greater detail, the user may choose another pattern (lab→vital→Beta Blockers 2→vital) to see the lab tests that these patients took. Visual interface 120 may show the patterns of Level 2. The patterns may indicate a trend, where Troponin T and Natriuretic Peptide are red, indicating the patients with these lab tests are more likely to be hospitalized. This is because these two lab tests are direct indicators of CHF and are usually associated with CHF patients with more severe conditions.

Advantageously, the present principles exploit the power of integrating pattern mining techniques with visualization to depict the relationships between medical events. It is noted that the present principles are much broader and are not limited to medical events. The insights derived from the present principles have been shown to match known expertise medical knowledge. The ability for physicians and clinical researchers to interactively explore frequent patterns using visually comprehensible interface shows great promise in supporting a better understanding of disease evolution and effective care pathways for patients.

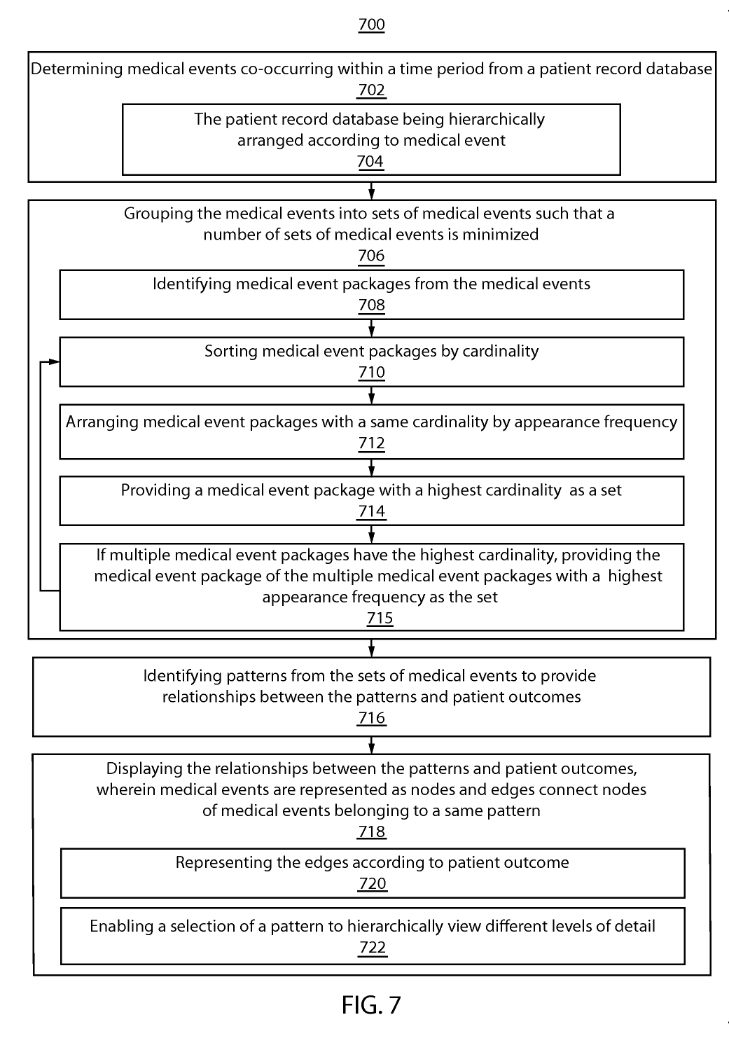

Referring now to FIG. 7, a block/flow diagram showing a method 700 for data analysis is illustratively depicted in accordance with one embodiment. In block 702, medical events co-occurring within a time period are determined from a patient record database. The time period may be, e.g., a day, such that the medical events co-occurring within the time period are Same Day Concurrent Events. The patient record database preferably includes a patient EMR indicating medical events and patient outcomes. Medical events may include, e.g., lab, vital, medication and diagnosis; however, other medical events are also contemplated. In block 704, the patient record database may be hierarchically arranged according to medical event.

In block 706, identified medical events are grouped into sets of medical events such that a number of sets of medical events is minimized. This may include applying a two-way sorting method to break down the identified medical events into regular and super events. In block 708, medical event packages are identified from the medical events. In block 710, medical event packages are sorted by cardinality. In block 712, medical event packages with a same cardinality are then arranged by appearance frequency. In block 714, the medical event package with a highest cardinality is provided as a set. If multiple medical event packages have the highest cardinality, in block 715, the medical event package of the multiple medical event packages with a highest appearance frequency is provided as the set. This process is repeated for remaining portions of the identified medical events. Advantageously, the number of events of the identified medical events is reduced.

In block 716, patterns from the sets of medical events are identified to provide relationships between patterns and patient outcomes. Preferably, the SPAM method is applied to the sets of medical events to identify patterns. Patterns may be collected into a dictionary and a bag-of-pattern (BOP) representation of each patient may be constructed. The BOP representation may include a vector with values corresponding to frequencies of the pattern.

In block 718, the relationships between the patterns and patient outcomes are displayed. Medical events may be represented as nodes and edges connect nodes of medical events belonging to a same pattern. In block 720, the edges are represented according to patient outcome. Preferably, edges are represented according to patient outcome by color. For example, positive patient outcomes can be represented by blue, negative patient outcomes can be represented by red and neutral patient outcomes can be represented by gray. Other representations are also contemplated, such as, e.g., patterns. In block 722, a selection of a pattern is enabled to hierarchically view different levels of detail. The hierarchical view may correspond to the hierarchy of the patient record database. Enabling a selection may include hovering over (e.g., mouse-over) edges to view additional information.

Having described preferred embodiments of a system and method for hierarchical exploration of longitudinal medical events (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

1. A method for data analysis, comprising:
determining medical events co-occurring within a time period from a patient record database;
grouping the medical events into sets of medical events such that a number of sets of medical events is minimized based upon medical event cardinality; and
identifying patterns from the sets of medical events, using a processor, to provide relationships between the patterns and patient outcomes.

2. The method as recited in claim 1, further comprising displaying the relationships between the patterns and patient outcomes.

3. The method as recited in claim 2, wherein displaying includes representing medical events as nodes and connecting nodes of medical events belonging to a same pattern with edges.

4. The method as recited in claim 3, further comprising representing edges according to patient outcome.

5. The method as recited in claim 3, further comprising enabling a selection of a node and/or pattern to hierarchically view different levels of detail.

6. The method as recited in claim 1, wherein grouping includes:
identifying one or more medical event packages with a highest cardinality from the medical events; and
providing a medical event package from the one or more medical event packages with a highest frequency of appearance as the set.

7. The method as recited in claim 1, wherein identifying patterns includes employing frequent pattern mining to identify patterns.

8. The method as recited in claim 1, wherein identifying patterns includes arranging patterns into a pattern dictionary.

9. The method as recited in claim 1, wherein identifying patterns includes representing patterns as a bag-of-patterns representation, which includes a vector having weights corresponding to pattern frequency.

10. The method as recited in claim 1, wherein the patient record database is hierarchically arranged according to medical event.

11-25. (canceled)
